(19) United States
(12) Patent Application Publication
(10) Pub. No.: US 2005/0209578 A1
(43) Pub. Date: Sep. 22, 2005

(54) Ultrasonic catheter with segmented fluid delivery

(76) Inventors: Edward Albert Christian Evans, Edmonds, WA (US); Robert L. Wilcox, Bothell, WA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/045,799

(22) Filed: Jan. 28, 2005

Related U.S. Application Data
(60) Provisional application No. 60/540,879, filed on Jan. 29, 2004.
(60) Provisional application No. 60/578,800, filed on Jun. 10, 2004.

(51) Int. Cl. A61M 31/00

(52) U.S. Cl. 604/500; 604/22

(57) ABSTRACT

An ultrasound catheter is configured to be positioned at a treatment site within a patient's vasculature. The catheter comprises an elongate tubular body forming a utility lumen. The catheter further comprises an ultrasound assembly configured to be movably positioned within the utility lumen. The ultrasound assembly includes a plurality of ultrasound radiating members. The catheter further comprises a plurality of fluid delivery lumens formed within the elongate tubular body. Each fluid delivery lumen includes one or more fluid delivery ports configured to allow a fluid to flow from within the fluid delivery lumen to the treatment site. A first fluid delivery lumen includes one or more fluid delivery ports over a first region of the tubular body. A second fluid delivery lumen includes one or more fluid delivery ports over a second region of the tubular body. The first region of the tubular body and the second region of the tubular body have different lengths.

### PRIORITY APPLICATION

This application claims the benefit of U.S. Provisional Application 60/540,879 (filed 29 Jan. 2004; Attorney Docket EKOS.171PR) and U.S. Provisional Application 60/578,800 (filed 10 Jun. 2004; Attorney Docket EKOS.174PR). Both of these priority applications are hereby incorporated by reference herein in their entirety.

### FIELD OF THE INVENTION

The present invention relates generally to treatment of vascular occlusions, and more specifically to treatment of vascular occlusions with ultrasonic energy and a therapeutic compound.

### BACKGROUND OF THE INVENTION

Several medical applications use ultrasonic energy. For example, U.S. Pat. Nos. 4,821,740, 4,953,565 and 5,007,438 disclose the use of ultrasonic energy to enhance the effect of various therapeutic compounds. An ultrasonic catheter can be used to deliver ultrasonic energy and a therapeutic compound to a treatment site within a patient's body. Such an ultrasonic catheter typically includes an ultrasound assembly configured to generate ultrasonic energy and a fluid delivery lumen for delivering the therapeutic compound to the treatment site.

As taught in U.S. Pat. No. 6,001,069, ultrasonic catheters can be used to treat human blood vessels that have become partially or completely occluded by plaque, thrombi, emboli or other substances that reduce the blood carrying capacity of the vessel. To remove or reduce the occlusion, the ultrasonic catheter is used to deliver solutions containing therapeutic compounds directly to the occlusion site. Ultrasonic energy generated by the ultrasound assembly enhances the effect of the therapeutic compounds. Such a device can be used in the treatment of diseases such as peripheral arterial occlusion or deep vein thrombosis. In such applications, the ultrasonic energy enhances treatment of the occlusion with therapeutic compounds such as urokinase, tissue plasminogen activator (“tPA”), recombinant tissue plasminogen activator (“rtPA”) and the like. The entire disclosure of U.S. Pat. No. 6,001,069 is incorporated by reference herein. Further information on enhancing the effect of a therapeutic compound using ultrasonic energy is provided in U.S. Pat. Nos. 5,318,014, 5,362,309, 5,474,531, 5,628,728 and 6,210,356.

Ultrasonic catheters can also be used to enhance gene therapy at a treatment site within the patient's body. For example, U.S. Pat. No. 6,135,976 discloses an ultrasonic catheter having one or more expandable sections capable of occluding a section of a body lumen, such as a blood vessel. A gene therapy composition is then delivered to the occluded vessel through the catheter fluid delivery lumen. Ultrasonic energy generated by the ultrasound assembly is applied to the occluded vessel, thereby enhancing the delivery of a genetic composition into the cells of the occluded vessel.

Ultrasonic catheters can also be used to enhance delivery and activation of light activated drugs. For example, U.S. Pat. No. 6,176,842 discloses methods for using an ultrasonic catheter to treat biological tissues by delivering a light activated drug to the biological tissues and exposing the light activated drug to ultrasound energy.

### SUMMARY OF THE INVENTION

Vessel occlusions can be large. For example, a deep vein thrombus in a patient's lower leg can have a length of 50 cm or more. Early treatment protocols for long occlusions used an infusion catheter to drip a lytic drug at one end of the occlusion; as the occlusion was dissolved, the catheter would be advanced. This process was repeated until the entire clot was dissolved. This treatment technique is extremely time-consuming. In an improved treatment technique, a therapeutic compound can be selectively delivered along the lateral dimension of an ultrasonic catheter, as disclosed herein. The catheter can be pushed through the clot, and therefore the therapeutic compound can be delivered at certain points within the occlusion, along a partial segment of the occlusion, or along the entire length of the occlusion.

In one embodiment of the present invention, an ultrasound catheter is configured to be positioned at a treatment site within a patient's vasculature. The catheter comprises an elongate tubular body forming a utility lumen. The catheter further comprises an ultrasound assembly configured to be movably positioned within the utility lumen. The ultrasound assembly includes a plurality of ultrasound radiating members. The catheter further comprises a plurality of fluid delivery lumens formed within the elongate tubular body. Each fluid delivery lumen includes one or more fluid delivery ports configured to allow a fluid to flow from within the fluid delivery lumen to the treatment site. A first fluid delivery lumen includes one or more fluid delivery ports over a first region of the tubular body. A second fluid delivery lumen includes one or more fluid delivery ports over a second region of the tubular body. The first region of the tubular body and the second region of the tubular body have different lengths.

In another embodiment of the present invention, an ultrasound catheter comprises a tubular body forming a utility lumen. The catheter further comprises an ultrasound assembly configured to be movably positioned within the utility lumen. The ultrasound assembly includes a plurality of ultrasound radiating members. The catheter further comprises a plurality of fluid delivery lumens formed within the tubular body. Each fluid delivery lumen includes one or more fluid delivery ports configured to allow a fluid to flow from within the delivery lumen to the treatment site. A first fluid delivery lumen includes one or more fluid delivery ports along a first region of the tubular body. A second fluid delivery lumen includes one or more fluid delivery ports along a second region of the tubular body. The first region includes a portion of the tubular body that is not included in the second region.

In another embodiment of the present invention, an apparatus comprises an elongate tubular body forming a utility lumen. The apparatus further comprises an ultrasound assembly configured to be movably positioned within the utility lumen. The apparatus further comprises a plurality of ultrasound radiating members positioned within the ultrasound assembly. The ultrasound radiating members are arranged into a electrical groups, such that a first group of the ultrasound radiating members can be separately activated with respect to a second group of the ultrasound radiating members. The apparatus further comprises a plurality of fluid delivery lumens formed within the tubular body and positioned around a circumference of the utility lumen. Each fluid delivery lumen includes one or more fluid delivery ports configured to allow a fluid to be expelled from the fluid delivery lumen. A first fluid delivery lumen includes one or more fluid delivery ports in a region of the tubular body where a second fluid delivery lumen includes no fluid delivery ports.

In another embodiment of the present invention, a method of treating a blockage within a patient's vasculature comprises positioning an ultrasound catheter at the treatment site. The method further comprises, in a first treatment phase, delivering a therapeutic compound and ultrasonic energy from a first portion of the ultrasound catheter. At least a portion of the blockage is exposed to the therapeutic compound and the ultrasonic energy. The delivery of therapeutic compound and ultrasonic energy is configured to reduce the blockage. The method further comprises monitoring progression of the blockage reduction. The method further comprises, in a second treatment phase, delivering a therapeutic compound and ultrasonic energy from a second portion of the ultrasound catheter. The second portion of the catheter includes a catheter region that is not included in the first portion of the catheter.

In another embodiment of the present invention, a catheter system for delivering ultrasonic energy and a therapeutic compound to a treatment site within a body lumen comprises an elongate tubular body having an energy delivery section. The tubular body defines a utility lumen. The system further comprises a fluid delivery lumen extending through at least a portion of the tubular body and having at least one fluid delivery port in the energy delivery section. The system further comprises an ultrasound assembly configured to be inserted into the utility lumen. The ultrasound assembly includes at least one ultrasound radiating member. The system further comprises a stiffening element positioned in the tubular body. The system further comprises a temperature sensor coupled to the stiffening element.

In another embodiment of the present invention, a catheter system comprises an elongate tubular body having an energy delivery section. The tubular body defines a utility lumen passing through the tubular body. The system further comprises a fluid delivery lumen extending through at least a portion of the tubular body and having at least one fluid delivery port in the energy delivery section. The system further comprises an ultrasound assembly configured for insertion into the utility lumen. The ultrasound assembly includes at least one ultrasound radiating member. The system further comprises a temperature sensor coupled to the elongate tubular body. The system further comprises a control box containing control circuitry to control the ultrasound radiating members based on signals received form the temperature sensor. The system further comprises an electrical connection between the tubular body and the ultrasound assembly. The electrical connection is configured to allow electronic signals to be passed between the tubular body and the ultrasound assembly.

In another embodiment of the present invention, a method for treating a blockage at a treatment site in a patient's vasculature comprises positioning an ultrasound catheter at the treatment site. The ultrasound catheter includes an elongate tubular body forming a utility lumen. The ultrasound catheter further includes a temperature sensor coupled to a stiffening element and to the tubular body. The ultrasound catheter further includes an ultrasound assembly configured to be movably positioned within the utility lumen. The ultrasound assembly includes a plurality of ultrasound radiating members. The ultrasound catheter further comprises a plurality of fluid delivery lumens formed within the elongate tubular body. Each fluid delivery lumen includes one or more fluid delivery ports configured to allow a fluid to flow from within the fluid delivery lumen to the treatment site. The method further comprises delivering ultrasonic energy from a first region of the ultrasound assembly to the treatment site. The method further comprises delivering a therapeutic compound through a first fluid delivery lumen to the treatment site. The region of therapeutic compound delivery and the region of ultrasonic energy delivery are overlapping. The method further comprises processing, in a control box coupled to the ultrasound assembly, a temperature signal collected from the temperature sensor. The method further comprises, in response to the collected temperature signal, delivering a therapeutic compound through a second fluid delivery lumen to the treatment site and delivering ultrasonic energy from a second region of the ultrasound assembly to the treatment site.

### BRIEF DESCRIPTION OF THE DRAWINGS

Exemplary embodiments of the vascular occlusion treatment system are illustrated in the accompanying drawings, which are for illustrative purposes only. The drawings comprise the following figures, in which like numerals indicate like parts.

FIG. 1 is a schematic illustration of an ultrasonic catheter configured for insertion into large vessels of the human body.

FIG. 2 is a cross-sectional view of the ultrasonic catheter of FIG. 1 taken along line 2-2.

FIG. 3 is a schematic illustration of an elongate inner core configured to be positioned within the central lumen of the catheter illustrated in FIG. 2.

FIG. 4 is a cross-sectional view of the elongate inner core of FIG. 3 taken along line 4-4.

FIG. 5 is a schematic wiring diagram illustrating an exemplary technique for electrically connecting five groups of ultrasound radiating members to form an ultrasound assembly.

FIG. 6 is a schematic wiring diagram illustrating an exemplary technique for electrically connecting one of the groups of FIG. 5.

FIG. 7A is a schematic illustration of the ultrasound assembly of FIG. 5 housed within the inner core of FIG. 4.

FIG. 7B is a cross-sectional view of the ultrasound assembly of FIG. 7A taken along line 7B-7B.

FIG. 7C is a cross-sectional view of the ultrasound assembly of FIG. 7A taken along line 7C-7C.

FIG. 7D is a side view of an ultrasound assembly center wire twisted into a helical configuration.

FIG. 8 illustrates the energy delivery section of the inner core of FIG. 4 positioned within the energy delivery section of the tubular body of FIG. 2.

FIG. 9 illustrates a wiring diagram for connecting a plurality of temperature sensors with a common wire.

FIG. 10 is a block diagram of a feedback control system for use with an ultrasonic catheter.

FIG. 11A is a side view of a treatment site.

FIG. 11B is a side view of the distal end of an ultrasonic catheter positioned at the treatment site of FIG. 11A.

FIG. 11C is a cross-sectional view of the distal end of the ultrasonic catheter of FIG. 11B positioned at the treatment site before a treatment.

FIG. 11D is a cross-sectional view of the distal end of the ultrasonic catheter of FIG. 11C, wherein an inner core has been inserted into the tubular body to perform a treatment.

FIG. 12 is a schematic diagram illustrating an exemplary ultrasonic catheter having fluid delivery lumens associated with fluid delivery ports along specific axial lengths of the ultrasonic catheter.

FIG. 12A is a schematic diagram illustrating flow rate over a length of a catheter.

FIG. 12B is a schematic illustration of a flow assembly configured to provide fluid to the fluid delivery lumens of the catheter of FIG. 12.

FIG. 12C is a schematic illustration of a modified flow assembly configured to provide fluid to the fluid delivery lumens of the catheter of FIG. 12.

FIG. 13A is a schematic diagram of a stiffening element having a temperature sensor coupled thereto.

FIG. 13B is a schematic diagram illustrating relative lengths of a stiffening element and a catheter body.

FIG. 13C is a schematic diagram illustrating a stiffening element having temperature sensors coupled thereto positioned within a catheter body.

FIG. 13D is a schematic diagram illustrating a seal used to couple a stiffening element to the catheter body 12.

FIG. 14A is a schematic diagram illustrating an exemplary embodiment of an ultrasonic catheter having two cables connecting the catheter to a control system.

FIG. 14B is a schematic diagram illustrating another embodiment of an ultrasonic catheter having a cable connecting the catheter to a control system.

### DETAILED DESCRIPTION OF PREFERRED EMBODIMENTS

As set forth above, methods and apparatuses have been developed that allow a vascular occlusion to be treated using both ultrasonic energy and a therapeutic compound having a controlled temperature. Disclosed herein are several exemplary embodiments of ultrasonic catheters that can be used to enhance the efficacy of therapeutic compounds at a treatment site within a patient's body. Also disclosed are exemplary methods for using such catheters. For example, as discussed in greater detail below, the ultrasonic catheters disclosed herein can be used to deliver a therapeutic compound having an elevated temperature, or to heat a therapeutic compound after it has been delivered at a treatment site within the patient's vasculature.

Introduction.

As used herein, the term “therapeutic compound” refers broadly, without limitation, and in addition to its ordinary meaning, to a drug, medicament, dissolution compound, genetic material or any other substance capable of effecting physiological functions. Additionally, a mixture includes substances such as these is also encompassed within this definition of “therapeutic compound”. Examples of therapeutic compounds include thrombolytic compounds, anti-thrombosis compounds, and other compounds used in the treatment of vascular occlusions, including compounds intended to prevent or reduce clot formation. In applications where human blood vessels that have become partially or completely occluded by plaque, thrombi, emboli or other substances that reduce the blood carrying capacity of a vessel, exemplary therapeutic compounds include, but are not limited to, heparin, urokinase, streptokinase, tPA, rtPA and BB-10153 (manufactured by British Biotech, Oxford, UK).

As used herein, the terms “ultrasonic energy”, “ultrasound” and “ultrasonic” refer broadly, without limitation, and in addition to their ordinary meaning, to mechanical energy transferred through longitudinal pressure or compression waves. Ultrasonic energy can be emitted as continuous or pulsed waves, depending on the parameters of a particular application. Additionally, ultrasonic energy can be emitted in waveforms having various shapes, such as sinusoidal waves, triangle waves, square waves, or other wave forms. Ultrasonic energy includes sound waves. In certain embodiments, the ultrasonic energy referred to herein has a frequency between about 20 kHz and about 20 MHz. For example, in one embodiment, the ultrasonic energy has a frequency between about 500 kHz and about 20 MHz. In another embodiment, the ultrasonic energy has a frequency between about 1 MHz and about 3 MHz. In yet another embodiment, the ultrasonic energy has a frequency of about 2 MHz. In certain embodiments described herein, the average acoustic power of the ultrasonic energy is between about 0.01 watts and 300 watts. In one embodiment, the average acoustic power is about 15 watts.

As used herein, the term “ultrasound radiating member” refers broadly, without limitation, and in addition to its ordinary meaning, to any apparatus capable of producing ultrasonic energy. An ultrasonic transducer, which converts electrical energy into ultrasonic energy, is an example of an ultrasound radiating member. An exemplary ultrasonic transducer capable of generating ultrasonic energy from electrical energy is a piezoelectric ceramic oscillator. Piezoelectric ceramics typically comprise a crystalline material, such as quartz, that changes shape when an electrical current is applied to the material. This change in shape, made oscillatory by an oscillating driving signal, creates ultrasonic sound waves. In other embodiments, ultrasonic energy can be generated by an ultrasonic transducer that is remote from the ultrasound radiating member, and the ultrasonic energy can be transmitted, via, for example, a wire that is coupled to the ultrasound radiating member.

In certain applications, the ultrasonic energy itself provides a therapeutic effect to the patient. Examples of such therapeutic effects include preventing or reducing stenosis and/or restenosis; tissue ablation, abrasion or disruption; promoting temporary or permanent physiological changes in intracellular or intercellular structures; and rupturing micro-balloons or micro-bubbles for therapeutic compound delivery. Further information about such methods can be found in U.S. Pat. Nos. 5,261,291 and 5,431,663.

The ultrasonic catheters described herein can be configured for application of ultrasonic energy over a substantial length of a body lumen, such as, for example, the larger vessels located in the leg. In other embodiments, the ultrasonic catheters described herein can be configured to be inserted into the small cerebral vessels, in solid tissues, in duct systems and in body cavities. Additional embodiments that can be combined with certain features and aspects of the embodiments described herein are described in U.S. patent application Ser. No. 10/291,891, filed 7 Nov. 2002, the entire disclosure of which is hereby incorporated herein by reference.

Overview of a Large Vessel Ultrasonic Catheter.

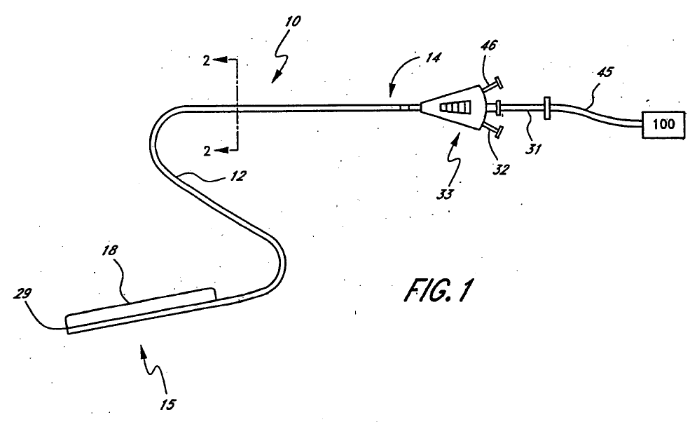

FIG. 1 schematically illustrates an ultrasonic catheter 10 configured for use in the large vessels of a patient's anatomy. For example, the ultrasonic catheter 10 illustrated in FIG. 1 can be used to treat long segment peripheral arterial occlusions, such as those in the vascular system of the leg.

As illustrated in FIG. 1, the ultrasonic catheter 10 generally includes a multi-component, elongate flexible tubular body 12 having a proximal region 14 and a distal region 15. The tubular body 12 includes a flexible energy delivery section 18 located in the distal region 15. The tubular body 12 and other components of the catheter 10 can be manufactured in accordance with a variety of techniques known to an ordinarily skilled artisan. Suitable materials and dimensions can be readily selected based on the natural and anatomical dimensions of the treatment site and on the desired percutaneous access site.

For example, in an exemplary embodiment, the tubular body proximal region 14 comprises a material that has sufficient flexibility, kink resistance, rigidity and structural support to push the energy delivery section 18 through the patient's vasculature to a treatment site. Examples of such materials include, but are not limited to, extruded polytetrafluoroethylene (“PTFE”), polyethylenes (“PE”), polyamides and other similar materials. In certain embodiments, the tubular body proximal region 14 is reinforced by braiding, mesh or other constructions to provide increased kink resistance and ability to be pushed. For example, nickel titanium or stainless steel wires can be placed along or incorporated into the tubular body 12 to reduce kinking.

For example, in an embodiment configured for treating thrombus in the arteries of the leg, the tubular body 12 has an outside diameter between about 0.060 inches and about 0.075 inches. In another embodiment, the tubular body 12 has an outside diameter of about 0.071 inches. In certain embodiments, the tubular body 12 has an axial length of approximately 105 centimeters, although other lengths can be used in other applications.

In an exemplary embodiment, the tubular body energy delivery section 18 comprises a material that is thinner than the material comprising the tubular body proximal region 14. In another exemplary embodiment, the tubular body energy delivery section 18 comprises a material that has a greater acoustic transparency than the material comprising the tubular body proximal region 14. Thinner materials generally have greater acoustic transparency than thicker materials. Suitable materials for the energy delivery section 18 include, but are not limited to, high or low density polyethylenes, urethanes, nylons, and the like. In certain modified embodiments, the energy delivery section 18 comprises the same material or a material of the same thickness as the proximal region 18.

In an exemplary embodiment, the tubular body 12 is divided into at least three sections of varying stiffness. The first section, which includes the proximal region 14, has a relatively higher stiffness. The second section, which is located in an intermediate region between the proximal region 14 and the distal region 15, has a relatively lower stiffness. This configuration further facilitates movement and placement of the catheter 10. The third section, which includes the energy delivery section 18, has a relatively lower stiffness than the second section in spite of the presence of ultrasound radiating members which can be positioned therein.

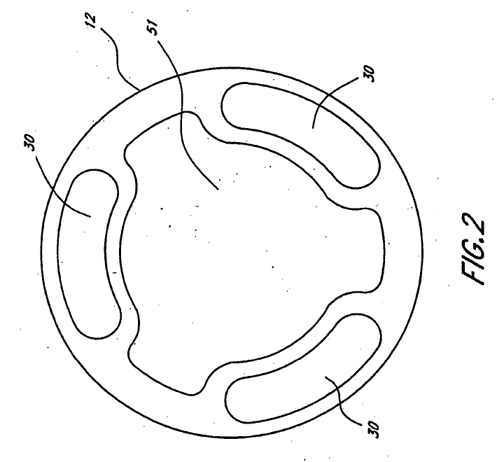

FIG. 2 illustrates a cross section of the tubular body 12 taken along line 2-2 in FIG. 1. In the embodiment illustrated in FIG. 2, three fluid delivery lumens 30 are incorporated into the tubular body 12. In other embodiments, more or fewer fluid delivery lumens can be incorporated into the tubular body 12. In such embodiments, the arrangement of the fluid delivery lumens 30 provides a hollow central lumen 51 passing through the tubular body 12. The cross-section of the tubular body 12, as illustrated in FIG. 2, is substantially constant along the length of the catheter 10. Thus, in such embodiments, substantially the same cross-section is present in both the proximal region 14 and the distal region 15 of the tubular body 12, including the energy delivery section 18.

In certain embodiments, the central lumen 51 has a minimum diameter greater than about 0.030 inches. In another embodiment, the central lumen 51 has a minimum diameter greater than about 0.037 inches. In an exemplary embodiment, the fluid delivery lumens 30 have dimensions of about 0.026 inches wide by about 0.0075 inches high, although other dimensions can be used in other embodiments.

In an exemplary embodiment, the central lumen 51 extends through the length of the tubular body 12. As illustrated in FIG. 1, the central lumen 51 has a distal exit port 29 and a proximal access port 31. The proximal access port 31 forms part of the backend hub 33, which is attached to the tubular body proximal region 14. In such embodiments, the backend hub also includes a cooling fluid fitting 46, which is hydraulically connected to the central lumen 51. In such embodiments, the backend hub 33 also includes a therapeutic compound inlet port 32, which is hydraulically coupled to the fluid delivery lumens 30, and which can also be hydraulically coupled to a source of therapeutic compound via a hub such as a Luer fitting.

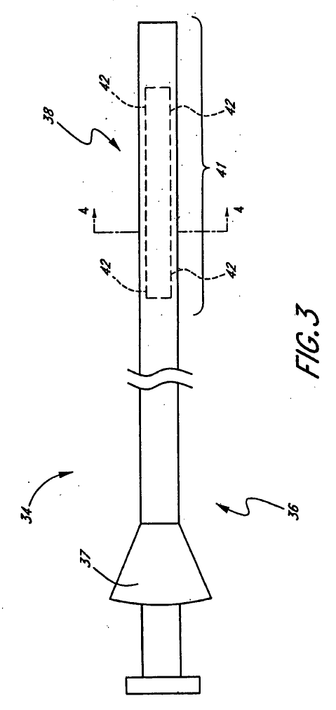

The central lumen 51 is configured to receive an elongate inner core 34, an exemplary embodiment of which is illustrated in FIG. 3. In such embodiments, the elongate inner core 34 includes a proximal region 36 and a distal region 38. A proximal hub 37 is fitted on one end of the inner core proximal region 36. One or more ultrasound radiating members 40 are positioned within an inner core energy delivery section 41 that is located within the distal region 38. The ultrasound radiating members 40 form an ultrasound assembly 42, which will be described in greater detail below.

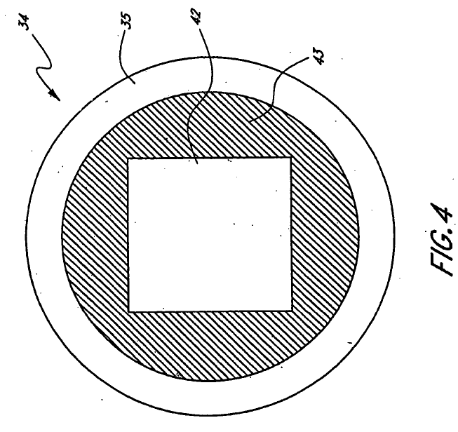

As shown in the cross-section illustrated in FIG. 4, which is taken along lines 4-4 in FIG. 3, in an exemplary embodiment, the inner core 34 has a cylindrical shape, with an outer diameter that permits the inner core 34 to be inserted into the central lumen 51 of the tubular body 12 via the proximal access port 31. Suitable outer diameters of the inner core 34 include, but are not limited to, between about 0.010 inches and about 0.100 inches. In another embodiment, the outer diameter of the inner core 34 is between about 0.020 inches and about 0.080 inches. In yet another embodiment, the inner core 34 has an outer diameter of about 0.035 inches.

Still referring to FIG. 4, the inner core 34 includes a cylindrical outer body 35 that houses the ultrasound assembly 42. The ultrasound assembly 42 includes wiring and ultrasound radiating members, described in greater detail in FIGS. 5 through 7D, such that the ultrasound assembly 42 is capable of radiating ultrasonic energy from the energy delivery section 41 of the inner core 34. The ultrasound assembly 42 is electrically connected to the backend hub 33, where the inner core 34 can be connected to a control system 100 via cable 45 (illustrated in FIG. 1). In an exemplary embodiment, an electrically insulating potting material 43 fills the inner core 34, surrounding the ultrasound assembly 42, thus reducing or preventing movement of the ultrasound assembly 42 with respect to the outer body 35. In one embodiment, the thickness of the outer body 35 is between about 0.0002 inches and 0.010 inches. In another embodiment, the thickness of the outer body 35 is between about 0.0002 inches and 0.005 inches. In yet another embodiment, the thickness of the outer body 35 is about 0.0005 inches.

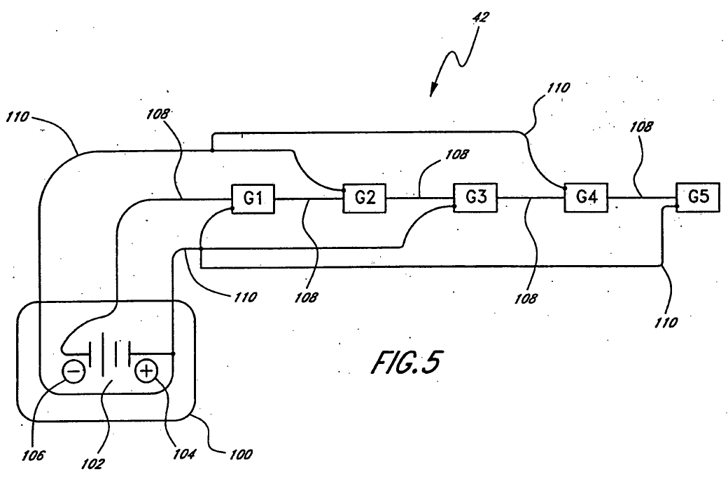

In an exemplary embodiment, the ultrasound assembly 42 includes a plurality of ultrasound radiating members 40 that are divided into one or more groups. For example, FIGS. 5 and 6 are schematic wiring diagrams illustrating one technique for connecting five groups of ultrasound radiating members 40 to form the ultrasound assembly 42. As illustrated in FIG. 5, the ultrasound assembly 42 comprises five groups G1, G2, G3, G4, G5 of ultrasound radiating members 40 that are electrically connected to each other. The five groups are also electrically connected to the control system 100.

Still referring to FIG. 5, in an exemplary embodiment, the control circuitry 100 includes a voltage source 102 having a positive terminal 104 and a negative terminal 106. The negative terminal 106 is connected to common wire 108, which connects the five groups G1-G5 of ultrasound radiating members 40 in series. The positive terminal 104 is connected to a plurality of lead wires 110, which each connect to one of the five groups G1-G5 of ultrasound radiating members 40. Thus, under this configuration, each of the five groups G1-G5, one of which is illustrated in FIG. 6, is connected to the positive terminal 104 via one of the lead wires 110, and to the negative terminal 106 via the common wire 108.

Referring now to FIG. 6, each group G1-G5 includes a plurality of ultrasound radiating members 40. Each of the ultrasound radiating members 40 is electrically connected to the common wire 108 and to the lead wire 110 via a positive contact wires 112. Thus, when wired as illustrated, a substantially constant voltage difference will be applied to each ultrasound radiating member 40 in the group. Although the group illustrated in FIG. 6 includes twelve ultrasound radiating members 40, in other embodiments, more or fewer ultrasound radiating members 40 can be included in the group. Likewise, more or fewer than five groups can be included within the ultrasound assembly 42 illustrated in FIG. 5.

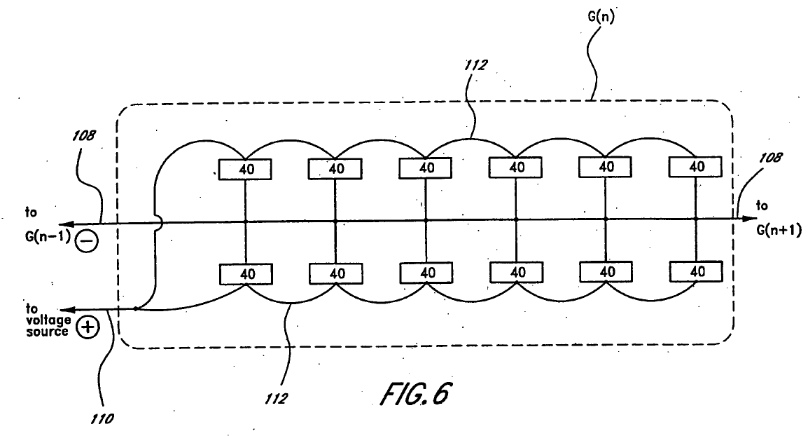

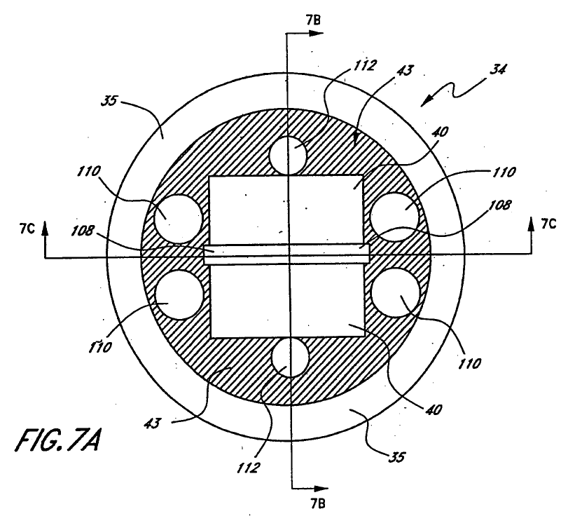

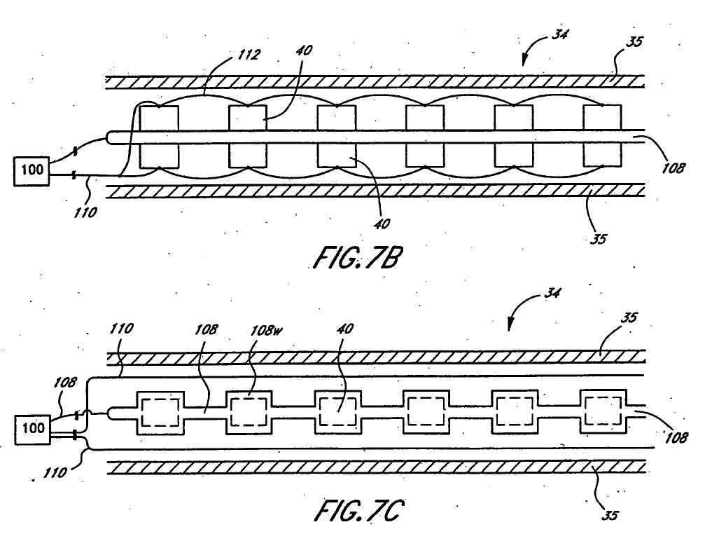

FIG. 7A illustrates an exemplary technique for arranging the components of the ultrasound assembly 42 (as schematically illustrated in FIG. 5) into the inner core 34 (as schematically illustrated in FIG. 4). FIG. 7A is a cross-sectional view of the ultrasound assembly 42 taken within group G1 in FIG. 5, as indicated by the presence of four lead wires 110. For example, if a cross-sectional view of the ultrasound assembly 42 was taken within group G4 in FIG. 5, only one lead wire 110 would be present (that is, the one lead wire connecting group G5).

In the exemplary embodiment illustrated in FIG. 7A, the common wire 108 includes an elongate, flat piece of electrically conductive material in electrical contact with a pair of ultrasound radiating members 40. Each of the ultrasound radiating members 40 is also in electrical contact with a positive contact wire 112. Because the common wire 108 is connected to the negative terminal 106, and the positive contact wire 112 is connected to the positive terminal 104, a voltage difference can be created across each ultrasound radiating member 40. In such embodiments, lead wires 110 are separated from the other components of the ultrasound assembly 42, thus preventing interference with the operation of the ultrasound radiating members 40 as described above. For example, in an exemplary embodiment, the inner core 34 is filled with an insulating potting material 43, thus deterring unwanted electrical contact between the various components of the ultrasound assembly 42.

FIGS. 7B and 7C illustrate cross sectional views of the inner core 34 of FIG. 7A taken along lines 7B-7B and 7C-7C, respectively. As illustrated in FIG. 7B, the ultrasound radiating members 40 are mounted in pairs along the common wire 108. The ultrasound radiating members 40 are connected by positive contact wires 112, such that substantially the same voltage is applied to each ultrasound radiating member 40. As illustrated in FIG. 7C, the common wire 108 includes wide regions 108W upon which the ultrasound radiating members 40 can be mounted, thus reducing the likelihood that the paired ultrasound radiating members 40 will short together. In certain embodiments, outside the wide regions 108W, the common wire 108 can have a more conventional, rounded wire shape.

In a modified embodiment, such as illustrated in FIG. 7D, the common wire 108 is twisted to form a helical shape before being fixed within the inner core 34. In such embodiments, the ultrasound radiating members 40 are oriented in a plurality of radial directions, thus enhancing the radial uniformity of the resulting ultrasonic energy field.

The wiring arrangement described above can be modified to allow each group G1, G2, G3, G4, G5 to be independently powered. Specifically, by providing a separate power source within the control system 100 for each group, each group can be individually turned on or off, or can be driven at an individualized power level. This advantageously allows the delivery of ultrasonic energy to be “turned off” in regions of the treatment site where treatment is complete, thus preventing deleterious or unnecessary ultrasonic energy to be applied to the patient.

The embodiments described above, and illustrated in FIGS. 5 through 7, include a plurality of ultrasound radiating members grouped spatially. That is, in such embodiments, the ultrasound radiating members within a certain group are positioned adjacent to each other, such that when a single group is activated, ultrasonic energy is delivered from a certain length of the ultrasound assembly. However, in modified embodiments, the ultrasound radiating members of a certain group may be spaced apart from each other, such that the ultrasound radiating members within a certain group are not positioned adjacent to each other. In such embodiments, when a single group is activated, ultrasonic energy can be delivered from a larger, spaced apart portion of the ultrasound assembly. Such modified embodiments can be advantageous in applications where a less focussed, more diffuse ultrasonic energy field is to be delivered to the treatment site.

In an exemplary embodiment, the ultrasound radiating members 40 comprise rectangular lead zirconate titanate (“PZT”) ultrasound transducers that have dimensions of about 0.017 inches by about 0.010 inches by about 0.080 inches. In other embodiments, other configurations and dimensions can be used. For example, disc-shaped ultrasound radiating members 40 can be used in other embodiments. In an exemplary embodiment, the common wire 108 comprises copper, and is about 0.005 inches thick, although other electrically conductive materials and other dimensions can be used in other embodiments. In an exemplary embodiment, lead wires 110 are 36 gauge electrical conductors, and positive contact wires 112 are 42 gauge electrical conductors. However, other wire gauges can be used in other embodiments.

As described above, suitable frequencies for the ultrasound radiating members 40 include, but are not limited to, from about 20 kHz to about 20 MHz. In one embodiment, the frequency is between about 500 kHz and about 20 MHz, and in another embodiment the frequency is between about 1 MHz and about 3 MHz. In yet another embodiment, the ultrasound radiating members 40 are operated with a frequency of about 2 MHz.

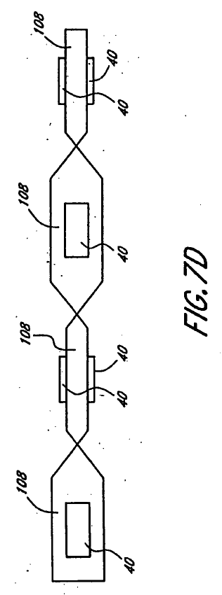

FIG. 8 illustrates the inner core 34 positioned within the tubular body 12. Details of the ultrasound assembly 42, provided in FIG. 7A, are omitted for clarity. As described above, the inner core 34 can be slid within the central lumen 51 of the tubular body 12, thereby allowing the inner core energy delivery section 41 to be positioned within the tubular body energy delivery section 18. For example, in an exemplary embodiment, the materials comprising the inner core energy delivery section 41, the tubular body energy delivery section 18, and the potting material 43 all comprise materials having a similar acoustic impedance, thereby minimizing ultrasonic energy losses across material interfaces.

FIG. 8 further illustrates placement of fluid delivery ports 58 within the tubular body energy delivery section 18. As illustrated, holes or slits are formed from the fluid delivery lumen 30 through the tubular body 12, thereby permitting fluid flow from the fluid delivery lumen 30 to the treatment site. A plurality of fluid delivery ports 58 can be positioned axially along the tubular body 12. Thus, a source of therapeutic compound coupled to the inlet port 32 provides a hydraulic pressure which drives the therapeutic compound through the fluid delivery lumens 30 and out the fluid delivery ports 58.

By spacing the fluid delivery lumens 30 around the circumference of the tubular body 12 substantially evenly, as illustrated in FIG. 8, a substantially uniform flow of therapeutic compound around the circumference of the tubular body 12 can be achieved. Additionally, the size, location and geometry of the fluid delivery ports 58 can be selected to provide uniform fluid flow from the fluid delivery ports 30 to the treatment site. For example, in one embodiment, fluid delivery ports closer to the proximal region of the energy delivery section 18 have smaller diameters than fluid delivery ports closer to the distal region of the energy delivery section 18, thereby allowing uniform delivery of therapeutic compound in the energy delivery section.

For example, in one embodiment in which the fluid delivery ports 58 have similar sizes along the length of the tubular body 12, the fluid delivery ports 58 have a diameter between about 0.0005 inches to about 0.0050 inches. In another embodiment in which the size of the fluid delivery ports 58 changes along the length of the tubular body 12, the fluid delivery ports 58 have a diameter between about 0.001 inches to about 0.005 inches in the proximal region of the energy delivery section 18, and between about 0.005 inches to about 0.0020 inches in the distal region of the energy delivery section 18. The increase in size between adjacent fluid delivery ports 58 depends on a variety of factors, including the material comprising the tubular body 12, and on the size of the fluid delivery lumen 30. The fluid delivery ports 58 can be created in the tubular body 12 by punching, drilling, burning or ablating (such as with a laser), or by other suitable methods. Therapeutic compound flow along the length of the tubular body 12 can also be increased by increasing the density of the fluid delivery ports 58 toward the distal region of the energy delivery section.

In certain applications, a spatially nonuniform flow of therapeutic compound from the fluid delivery ports 58 to the treatment site is to be provided. In such applications, the size, location and geometry of the fluid delivery ports 58 can be selected to provide such nonuniform fluid flow.

Referring still to FIG. 8, placement of the inner core 34 within the tubular body 12 further defines cooling fluid lumens 44. Cooling fluid lumens 44 are formed between an outer surface 39 of the inner core 34 and an inner surface 16 of the tubular body 12. In certain embodiments, a cooling fluid is introduced through the proximal access port 31 such that cooling fluid flows through cooling fluid lumens 44 and out of the catheter 10 through distal exit port 29 (see FIG. 1). In an exemplary embodiment, the cooling fluid lumens 44 are substantially evenly spaced around the circumference of the tubular body 12 (that is, at approximately 120° increments for a three-lumen configuration), thereby providing substantially uniform cooling fluid flow over the inner core 34. Such a configuration advantageously removes thermal energy from the treatment site. As will be explained below, the flow rate of the cooling fluid and the power to the ultrasound assembly 42 can be adjusted to maintain the temperature of the inner core energy delivery section 41, or of the treatment site generally, within a desired range.

In an exemplary embodiment, the inner core 34 can be rotated or moved within the tubular body 12. Specifically, movement of the inner core 34 can be accomplished by maneuvering the proximal hub 37 while holding the backend hub 33 stationary. The inner core outer body 35 is at least partially constructed from a material that provides enough structural support to permit movement of the inner core 34 within the tubular body 12 without kinking of the tubular body 12. Additionally, in an exemplary embodiment, the inner core outer body 35 comprises a material having the ability to transmit torque. Suitable materials for the inner core outer body 35 include, but are not limited to, polyimides, polyesters, polyurethanes, thermoplastic elastomers and braided polyimides.

In an exemplary embodiment, the fluid delivery lumens 30 and the cooling fluid lumens 44 are open at the distal end of the tubular body 12, thereby allowing the therapeutic compound and the cooling fluid to pass into the patient's vasculature at the distal exit port 29. In a modified embodiment, the fluid delivery lumens 30 can be selectively occluded at the distal end of the tubular body 12, thereby providing additional hydraulic pressure to drive the therapeutic compound out of the fluid delivery ports 58. In either configuration, the inner core 34 can be prevented from passing through the distal exit port 29 by providing the inner core 34 with a length that is less than the length of the tubular body 12. In other embodiments, a protrusion is formed within the tubular body 12 in the distal region 15, thereby preventing the inner core 34 from passing through the distal exit port 29.

In other embodiments, the catheter 10 includes an occlusion device positioned at the distal exit port 29. In such embodiments, the occlusion device has a reduced inner diameter that can accommodate a guidewire, but that is less than the inner diameter of the central lumen 51. Thus, the inner core 34 is prevented from extending past the occlusion device and out the distal exit port 29. For example, suitable inner diameters for the occlusion device include, but are not limited to, between about 0.005 inches and about 0.050 inches. In other embodiments, the occlusion device has a closed end, thus preventing cooling fluid from leaving the catheter 10, and instead recirculating to the tubular body proximal region 14. These and other cooling fluid flow configurations permit the power provided to the ultrasound assembly 42 to be increased in proportion to the cooling fluid flow rate. Additionally, certain cooling fluid flow configurations can reduce exposure of the patient's body to cooling fluids.

In an exemplary embodiment, such as illustrated in FIG. 8, the tubular body 12 includes one or more temperature sensors 20 that are positioned within the energy delivery section 18. In such embodiments, the tubular body proximal region 14 includes a temperature sensor lead which can be incorporated into cable 45 (illustrated in FIG. 1). Suitable temperature sensors include, but are not limited to, temperature sensing diodes, thermistors, thermocouples, resistance temperature detectors (“RTDs”) and fiber optic temperature sensors which use thermalchromic liquid crystals. Suitable temperature sensor 20 geometries include, but are not limited to, a point, a patch or a stripe. The temperature sensors 20 can be positioned within one or more of the fluid delivery lumens 30, and/or within one or more of the cooling fluid lumens 44.

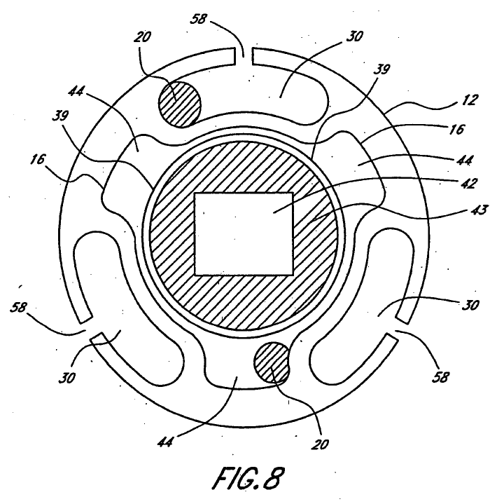

FIG. 9 illustrates an exemplary embodiment for electrically connecting the temperature sensors 20. In such embodiments, each temperature sensor 20 is coupled to a common wire 61 and is associated with an individual return wire 62. Accordingly, n+1 wires are passed through the tubular body 12 to independently sense the temperature at n temperature sensors 20. The temperature at a selected temperature sensor 20 can be determined by closing a switch 64 to complete a circuit between the return wire 62 associated with the selected thermocouple and the common wire 61. In embodiments wherein the temperature sensors 20 are thermocouples, the temperature can be calculated from the voltage in the circuit using, for example, a sensing circuit 63, which can be located within the external control circuitry 100.

In other embodiments, the temperature sensors 20 can be independently wired. In such embodiments, 2n wires are passed through the tubular body 12 to independently sense the temperature at n temperature sensors 20. In still other embodiments, the flexibility of the tubular body 12 can be improved by using fiber optic based temperature sensors 20. In such embodiments, flexibility can be improved because only n fiber optic members are used to sense the temperature at n independent temperature sensors 20.

FIG. 10 schematically illustrates one embodiment of a feedback control system 68 that can be used with the catheter 10. The feedback control system 68 can be integrated into the control system 100 that is connected to the inner core 34 via cable 45 (as illustrated in FIG. 1). The feedback control system 68 allows the temperature at each temperature sensor 20 to be monitored and allows the output power of the energy source 70 to be adjusted accordingly. A physician can, if desired, override the closed or open loop system.

In an exemplary embodiment, the feedback control system 68 includes an energy source 70, power circuits 72 and a power calculation device 74 that is coupled to the ultrasound radiating members 40. A temperature measurement device 76 is coupled to the temperature sensors 20 in the tubular body 12. A processing unit 78 is coupled to the power calculation device 74, the power circuits 72 and a user interface and display 80.

In an exemplary method of operation, the temperature at each temperature sensor 20 is determined by the temperature measurement device 76. The processing unit 78 receives each determined temperature from the temperature measurement device 76. The determined temperature can then be displayed to the user at the user interface and display 80.

In an exemplary embodiment, the processing unit 78 includes logic for generating a temperature control signal. The temperature control signal is proportional to the difference between the measured temperature and a desired temperature. The desired temperature can be determined by the user (as set at the user interface and display 80) or can be preset within the processing unit 78.

In such embodiments, the temperature control signal is received by the power circuits 72. The power circuits 72 are configured to adjust the power level, voltage, phase and/or current of the electrical energy supplied to the ultrasound radiating members 40 from the energy source 70. For example, when the temperature control signal is above a particular level, the power supplied to a particular group of ultrasound radiating members 40 is reduced in response to that temperature control signal. Similarly, when the temperature control signal is below a particular level, the power supplied to a particular group of ultrasound radiating members 40 is increased in response to that temperature control signal. After each power adjustment, the processing unit 78 monitors the temperature sensors 20 and produces another temperature control signal which is received by the power circuits 72.

In an exemplary embodiment, the processing unit 78 optionally includes safety control logic. The safety control logic detects when the temperature at a temperature sensor 20 exceeds a safety threshold. In this case, the processing unit 78 can be configured to provide a temperature control signal which causes the power circuits 72 to stop the delivery of energy from the energy source 70 to that particular group of ultrasound radiating members 40.

Because, in certain embodiments, the ultrasound radiating members 40 are mobile relative to the temperature sensors 20, it can be unclear which group of ultrasound radiating members 40 should have a power, voltage, phase and/or current level adjustment. Consequently, each group of ultrasound radiating members 40 can be identically adjusted in certain embodiments. For example, in a modified embodiment, the power, voltage, phase, and/or current supplied to each group of ultrasound radiating members 40 is adjusted in response to the temperature sensor 20 which indicates the highest temperature. Making voltage, phase and/or current adjustments in response to the temperature sensed by the temperature sensor 20 indicating the highest temperature can reduce overheating of the treatment site.

The processing unit 78 can also be configured to receive a power signal from the power calculation device 74. The power signal can be used to determine the power being received by each group of ultrasound radiating members 40. The determined power can then be displayed to the user on the user interface and display 80.

As described above, the feedback control system 68 can be configured to maintain tissue adjacent to the energy delivery section 18 below a desired temperature. For example, in certain applications, tissue at the treatment site is to have a temperature increase of less than or equal to approximately 6° C. As described above, the ultrasound radiating members 40 can be electrically connected such that each group of ultrasound radiating members 40 generates an independent output. In certain embodiments, the output from the power circuit maintains a selected energy for each group of ultrasound radiating members 40 for a selected length of time.

The processing unit 78 can comprise a digital or analog controller, such as a computer with software. In embodiments wherein the processing unit 78 is a computer, the computer can include a central processing unit (“CPU”) coupled through a system bus. In such embodiments, the user interface and display 80 can include a mouse, a keyboard, a disk drive, a display monitor, a nonvolatile memory system, and/or other computer components. In an exemplary embodiment, program memory and/or data memory is also coupled to the bus.

In another embodiment, in lieu of the series of power adjustments described above, a profile of the power to be delivered to each group of ultrasound radiating members 40 can be incorporated into the processing unit 78, such that a preset amount of ultrasonic energy to be delivered is pre-profiled. In such embodiments, the power delivered to each group of ultrasound radiating members 40 is provided according to the preset profiles.

In an exemplary embodiment, the ultrasound radiating members are operated in a pulsed mode. For example, in one embodiment, the time average power supplied to the ultrasound radiating members is between about 0.1 watts and about 2 watts. In another embodiment, the time average power supplied to the ultrasound radiating members is between about 0.5 watts and about 1.5 watts. In yet another embodiment, the time average power supplied to the ultrasound radiating members is approximately 0.6 watts or approximately 1.2 watts. In an exemplary embodiment, the duty cycle is between about 1% and about 50%. In another embodiment, the duty cycle is between about 5% and about 25%. In yet another embodiment, the duty cycles is approximately 7.5% or approximately 15%. In an exemplary embodiment, the pulse averaged power is between about 0.1 watts and about 20 watts. In another embodiment, the pulse averaged power is between approximately 5 watts and approximately 20 watts. In yet another embodiment, the pulse averaged power is approximately 8 watts or approximately 16 watts. The amplitude during each pulse can be constant or varied.

In an exemplary embodiment, the pulse repetition rate is between about 5 Hz and about 150 Hz. In another embodiment, the pulse repetition rate is between about 10 Hz and about 50 Hz. In yet another embodiment, the pulse repetition rate is approximately 30 Hz. In an exemplary embodiment, the pulse duration is between about 1 millisecond and about 50 milliseconds. In another embodiment, the pulse duration is between about 1 millisecond and about 25 milliseconds. In yet another embodiment, the pulse duration is approximately 2.5 milliseconds or approximately 5 milliseconds.

For example, in one particular embodiment, the ultrasound radiating members are operated at an average power of approximately 0.6 watts, a duty cycle of approximately 7.5%, a pulse repetition rate of approximately 30 Hz, a pulse average electrical power of approximately 8 watts and a pulse duration of approximately 2.5 milliseconds.

In an exemplary embodiment, the ultrasound radiating member used with the electrical parameters described herein has an acoustic efficiency greater than approximately 50%. In another embodiment, the ultrasound radiating member used with the electrical parameters described herein has an acoustic efficiency greater than approximately 75%. As described herein, the ultrasound radiating members can be formed in a variety of shapes, such as, cylindrical (solid or hollow), flat, bar, triangular, and the like. In an exemplary embodiment, the length of the ultrasound radiating member is between about 0.1 cm and about 0.5 cm, and the thickness or diameter of the ultrasound radiating member is between about 0.02 cm and about 0.2 cm.

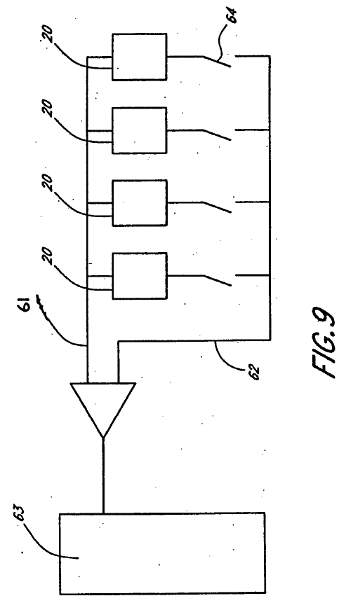

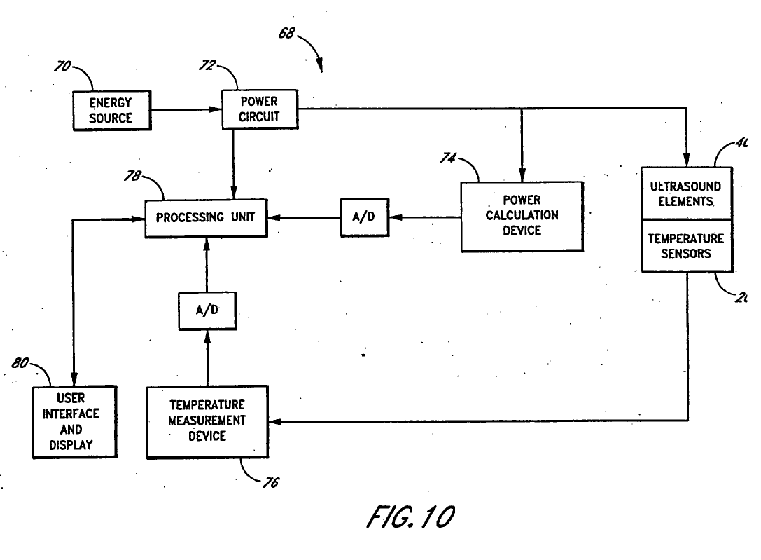

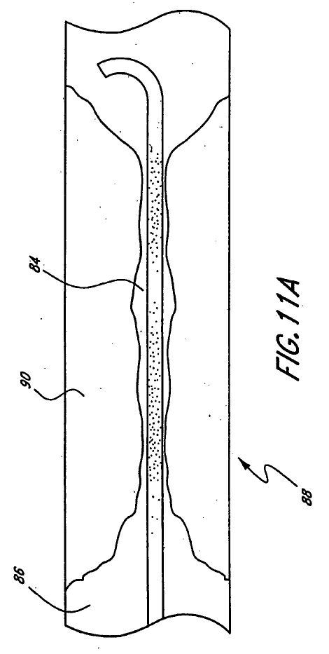

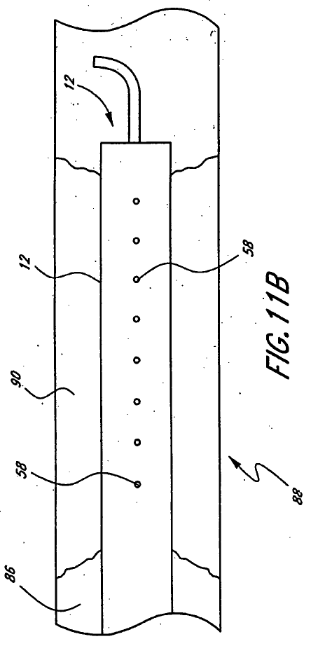

FIGS. 11A through 11D illustrate an exemplary method for using certain embodiments of the ultrasonic catheter 10 describe herein. As illustrated in FIG. 11A, a guidewire 84 similar to a guidewire used in typical angioplasty procedures is directed through a patient's vessels 86 to a treatment site 88 that includes a clot 90. The guidewire 84 is optionally directed through the clot 90. Suitable vessels 86 include, but are not limited to, the large periphery blood vessels of the body. Additionally, as mentioned above, the ultrasonic catheter 10 also has utility in various imaging applications or in applications for treating and/or diagnosing other diseases in other body parts.

As illustrated in FIG. 11B, the tubular body 12 is slid over and is advanced along the guidewire 84, for example using conventional over-the-guidewire techniques. The tubular body 12 is advanced until the energy delivery section 18 is positioned at the clot 90. In certain embodiments, radiopaque markers (not shown) are optionally positioned along the tubular body energy delivery section 18 to aid in the positioning of the tubular body 12 within the treatment site 88.

As illustrated in FIG. 11C, after the tubular body 12 is delivered to the treatment site 88, the guidewire 84 is withdrawn from the tubular body 12 by pulling the guidewire 84 from the proximal region 14 of the catheter 10 while holding the tubular body 12 stationary. This leaves the tubular body 12 positioned at the treatment site 88.

As illustrated in FIG. 11D, the inner core 34 is then inserted into the tubular body 12 until the ultrasound assembly 42 is positioned at least partially within the energy delivery section 18. In one embodiment, the ultrasound assembly 42 can be configured to be positioned at least partially within the energy delivery section 18 when the inner core 24 abuts the occlusion device at the distal end of the tubular body 12. Once the inner core 34 is positioned in such that the ultrasound assembly 42 is at least partially within the energy delivery section, the ultrasound assembly 42 is activated to deliver ultrasonic energy to the clot 90. As described above, in one embodiment, ultrasonic energy having a frequency between about 20 kHz and about 20 MHz is delivered to the treatment site.

In an exemplary embodiment, the ultrasound assembly 42 includes sixty ultrasound radiating members 40 spaced over a length of approximately 30 to approximately 50 cm. In such embodiments, the catheter 10 can be used to treat an elongate clot 90 without requiring moving or repositioning the catheter 10 during the treatment. However, in modified embodiments, the inner core 34 can be moved or rotated within the tubular body 12 during the treatment. Such movement can be accomplished by maneuvering the proximal hub 37 of the inner core 34 while holding the backend hub 33 stationary.

Still referring to FIG. 11D, arrows 48 indicate that a cooling fluid can be delivered through the cooling fluid lumen 44 and out the distal exit port 29. Likewise, arrows 49 indicate that a therapeutic compound can be delivered through the fluid delivery lumen 30 and out the fluid delivery ports 58 to the treatment site 88.

The cooling fluid can be delivered before, after, during or intermittently with the delivery of ultrasonic energy. Similarly, the therapeutic compound can be delivered before, after, during or intermittently with the delivery of ultrasonic energy. Consequently, the methods illustrated in FIGS. 11A through 11D can be performed in a variety of different orders than that described above. In an exemplary embodiment, the therapeutic compound and ultrasonic energy are delivered until the clot 90 is partially or entirely dissolved. Once the clot 90 has been sufficiently dissolved, the tubular body 12 and the inner core 34 are withdrawn from the treatment site 88.

Overview of Ultrasound Catheter with Treatment Sub-Regions.

As described above, and as illustrated in FIG. 2, in certain embodiments a plurality of fluid delivery lumens 30 are incorporated into the tubular body 12. As illustrated in FIG. 8, in certain embodiments the fluid delivery lumens 30 include fluid delivery ports 58 in the treatment region of the tubular body 12, thereby allowing a fluid within the lumens 30 to be delivered to the exterior of the catheter via the ports 58. As described above, in one embodiment, the lumens 30 are occluded at the distal end of the tubular body 12 and are used to deliver a therapeutic compound to a treatment region through a plurality of fluid delivery ports 58. In one embodiment, the delivery fluid delivery ports 58 for each lumen 30 generally extend along the entire treatment region such that the fluid delivery ports 58 for each lumen 30 generally occupy the same axial area of the catheter. As described above, within the treatment region, the size, location and geometry of the fluid delivery ports 58 is selected to provide uniform or nonuniform fluid flow from the fluid delivery ports 58 in the axial direction or a circumferential direction along the treatment region.

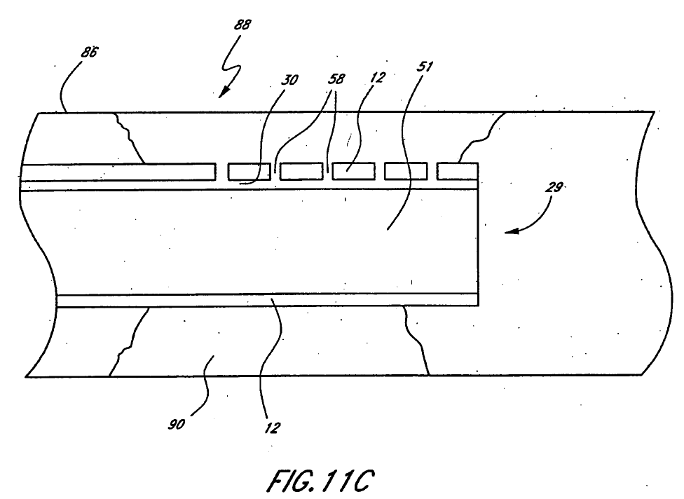

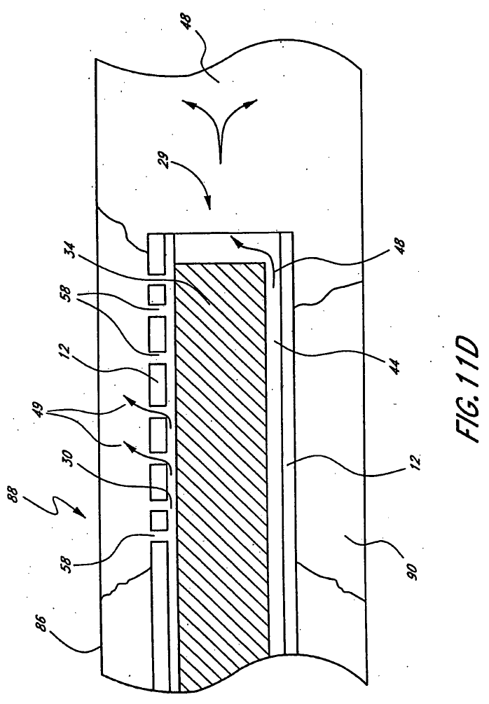

FIG. 12 illustrates a modified embodiment in which the treatment region is divided into treatment sub-regions. In the illustrated exemplary embodiment, the tubular body 12 is subdivided into three sub-regions A, B and C. Although the sub-regions are illustrated as being the same length in FIG. 12, they need not have the same length in other embodiments. Furthermore, more than or fewer than three treatment sub-regions can be used in other embodiments.

In one embodiment, the catheter is configured such that fluid delivery is controllable between the sub-regions. In the illustrated embodiment, fluid control between the sub-regions is accomplished by using the three fluid delivery lumens—A, B and C—incorporated into the interior of the tubular body. In such embodiments, fluid delivery lumen A has fluid delivery ports 56a in region A of the tubular body, fluid delivery lumen B has fluid delivery ports 56b in region B of the tubular body, and fluid delivery lumen C has fluid delivery ports 56c in region C of the tubular body. By passing a therapeutic compound along a selected fluid delivery lumen A, B or C, this configuration allows a therapeutic compound to be delivered along selected axial regions of the tubular body 12.

FIG. 12A illustrates an embodiment in which the fluid delivery ports 56a, 56b, 56c in each region A, B, C are configured to provide a non-uniform flow profile with respect to the length of the catheter. In particular, in the illustrated embodiment, the non-uniform flow profile is characterized by a “humped” profile in which the flow is biased towards the middle of each flow region, A, B, C. As shown, the profiles in each region may overlap; however, in a modified embodiment, they need not. When the catheter is embedded in a clot. This arrangement advantageously results in a more uniform flow profile over the length of the catheter as compared to, for example, an arrangement in which the flow is distributed evenly within each flow region, A, B, C.

In a modified embodiment, different therapeutic compounds are passed through different fluid delivery lumens. For example, in one embodiment a first therapeutic compound is delivered to one or more end portions of a vascular blockage (e.g. regions A and C), such as a proximal end and a distal end of the vascular blockage. Similarly, a second therapeutic compound is delivered to an internal portion if the vascular blockage (e.g., region B). Such a configuration is particularly useful where it is determined that the first therapeutic compound is more effective at treating an end portion of the vascular blockage, and the second therapeutic compound is more effective at treating an internal portion of the vascular blockage. In another embodiment, the second (or first) therapeutic compound may activate or react with the first (or second) therapeutic compound to create the desired therapeutic affect.

In another modified embodiment, the catheter is configured with more than or fewer than three treatment sub-regions. In such embodiments, the catheter optionally includes more than or fewer than three fluid delivery lumens with the fluid delivery ports of each lumen being associated with a specific sub-region. For example, in one such embodiment, a catheter includes four fluid delivery lumens, each configured to deliver a therapeutic compound to one of four treatment regions.

In yet another modified embodiment, one or more of the fluid delivery lumens is configured to have fluid delivery ports in more than one treatment sub-region. For example, in one such embodiment, a catheter with three delivery lumens and four treatment regions includes a delivery lumen that is configured to deliver therapeutic compound to more than one treatment region.

In yet another modified embodiment, the number of sub-regions along the tubular body is greater than or less than the number of fluid delivery lumens incorporated into the tubular body. For example, in one such embodiment, a catheter has two treatment regions and three delivery lumens. This configuration provides one dedicated delivery lumen for each of the treatment regions, as well as providing a delivery lumen capable of delivering a therapeutic compound to both treatment regions simultaneously.

In the embodiments disclosed herein, the delivery lumens optionally extend to the distal end of the catheter. For example, in one embodiment, a delivery lumen is configured to deliver a therapeutic compound to a proximal end of the vascular blockage does not extend to the distal end of the catheter.

In one embodiment, an tubular body has a treatment region of length 3n cm that is divided into three regions, each of length n cm. The tubular body has three fluid delivery lumens incorporated therein. A first fluid delivery lumen contains fluid delivery ports along the first region for a total of n cm of fluid delivery ports. A second fluid delivery lumen contains fluid delivery ports along the first and second regions for a total of 2n cm of fluid delivery ports. A third fluid delivery lumen contains fluid delivery ports along all 3n cm of the tubular body treatment region. Therapeutic compound can be delivered through one, two, or all three of the fluid delivery lumens depending on the length of the occlusion to be treated. In one such embodiment, n=6.

In another embodiment, the first treatment sub-region of the tubular body is 24 cm long, the second treatment sub-region of the tubular body is 8 cm long, and the third treatment sub-region of the tubular body is 8 cm long. In this embodiment, the treatment region of the tubular body is 40 cm long, and an ultrasound assembly capable of delivering ultrasonic energy along a 40 cm length is passed through the central lumen of the tubular body. In still another embodiment, the first sub-region of the tubular body is 20 cm long, the second sub-region of the tubular body is 10 cm long, and the third sub-region of the tubular body is 10 cm long. In this embodiment, the treatment region of the tubular body is 40 cm long, and an ultrasound assembly capable of delivering ultrasonic energy along a 40 cm length is passed through the central lumen of the tubular body.

The dimensions of the treatment regions and the fluid delivery lumens provided herein are approximate. Other lengths for fluid delivery lumens and treatment regions can be used in other embodiments.

The ultrasound assembly has a length that may be shorter than, longer than, or equal to a length of one the treatment regions A, B, C, in the tubular body 12. For example, in one embodiment the length of the ultrasound assembly is an integral multiple of length of an ultrasound radiating member group, as illustrated in FIGS. 5 and 6. In one embodiment, the length of an ultrasound radiating member group is approximately 6 cm, and the length of a treatment region A, B, C in the tubular body is also 6 cm. In another embodiment, the length of the tubular body treatment regions is an integral multiple of the length of an ultrasound radiating member group. For example, in one such embodiment the ultrasound radiating member groups are 6 cm long, and the tubular body treatment regions A, B, C are 12 cm long. In such embodiments, there is optionally more than one ultrasound radiating member group associated with each tubular body treatment region A, B, C.

An ultrasonic catheter with fluid delivery sub-regions is particularly advantageous in embodiments wherein an the occlusion to be treated is elongated. For example, in one application, a therapeutic compound is delivered to a selected sub-region of the occlusion. Thus, if treatment progresses faster in a particular sub-region of the occlusion, the therapeutic compound and ultrasonic energy delivered to that region of the occlusion can be selectively reduced or terminated, and the treatment can move to other regions of the occlusion.

An ultrasonic catheter with fluid delivery sub-regions can be used to treat occlusions having a wide variety of different lengths. For example, to treat a relatively short occlusion, a distal portion of the tubular body is delivered to the treatment site, and therapeutic compound is passed through a fluid delivery lumen having fluid delivery sub-regions in the distal portion of the tubular body. This same catheter can also be used to treat a relatively long occlusion by using more of the flow regions. In this manner, a single tubular body can be used to treat different lengths of occlusions, thereby reducing inventory costs. Additionally, the ultrasound radiating member groups of ultrasonic assembly are optionally configured to correspond to the fluid delivery sub-regions. In this manner, ultrasonic energy is selectively applied to the sub-regions that are positioned in or adjacent to the occlusion. Thus, in such embodiments, a single ultrasonic assembly and a single drug delivery catheter are used to treat occlusions of different lengths.

FIG. 12B illustrates one embodiment of a flow assembly 400 configured such that a surgeon can select which regions A, B, C will receive drugs. The flow assembly 400 comprises a valve assembly 402. The valve assembly is configured to receive therapeutic compound from an inlet conduit 404 and to selectively direct the therapeutic compound from the inlet conduit 404 to outlet conduits 406a, 406b, 406c. The outlet conduits 406a, 406b, 406c are, in turn, in communication with the flow regions A, B, C. An optional second inlet conduit 404 may be provided for receiving a second therapeutic compound. The valve assembly 402 may comprise any of a variety of flow control devices configured to selectively direct flow to a plurality of outlets. In one embodiment, the assembly 402 is provided with one or more pinch valves configured to pinch off flow lumens corresponding to the flow regions through which fluid delivery is to be reduced or eliminated. In such an embodiment, a “pinch-off” apparatus is controlled by a manual, pneumatic or electronic mechanism. Other embodiments may comprise any of a variety of combinations of three-way valves, rotary valves, trumpet valves, individual shut-off valves, roller-pumps and the like.

In one embodiment, the number and lengths of the treatment regions A, B, C is chosen based upon the observed or calculated distribution of occlusion lengths in the patient population. That is, number and lengths of the sub-region are chosen to correspond to common occlusion lengths in many patients. In a similar manner, the number and lengths of the ultrasound radiating members is also optionally configured to correspond to common occlusion lengths.

FIG. 12C illustrates another exemplary embodiment of a flow assembly 420. In this embodiment, the flow assembly 420 is configured to provide for uniform or substantially uniform delivery of therapeutic compound to the treatment site, despite, non-uniformities of the clot at the treatment site. The assembly 420 includes a flow control infusion pump 422, which is configured to deliver a substantially uniform rate of therapeutic compound. The pump 422 is connected to a valve 424 (e.g., a rotating valve), which has outlets 426a, 426b, 426c connected to the flow regions A, B, C. The valve 420 may be configured to sequentially delivery fluid to each of the outlets 426a, 426b, 426c while the flow to the other outlets are restricted or closed. In this manner, the delivery each flow region A, B, C, may be kept constant regardless of the local resistance or pressure at the drug ports within each region A, B, C. Thus, even though one flow region A, B, C, is subjected to a higher flow resistance due to, for example, the presence of more or denser clot material, the flow assembly 420 still provides a substantially uniform flow of therapeutic compounds to all of the flow regions A, B, C. This configuration advantageously prevents flow from being preferentially delivered to fluid delivery ports with the least external resistive hydraulic pressure.

In the embodiments described above, by controlling flow into the treatment sub-regions, non-uniform flow is delivered to the treatment site in the patient's vasculature. In some embodiments, the amount of flow delivered to each treatment sub-region is configured so as to produce improved treatment results for a given occlusion length. Additionally, the flow within each treatment sub-region is optionally manipulated by configuring the size, location and/or geometry of the fluid delivery ports to achieve uniform or non-uniform flow delivery within the treatment sub-region. Such techniques are optionally combined with selective electronic control of the ultrasound radiating member groups within treatment sub-regions.

Overview of Ultrasound Catheter with Temperature Sensors.

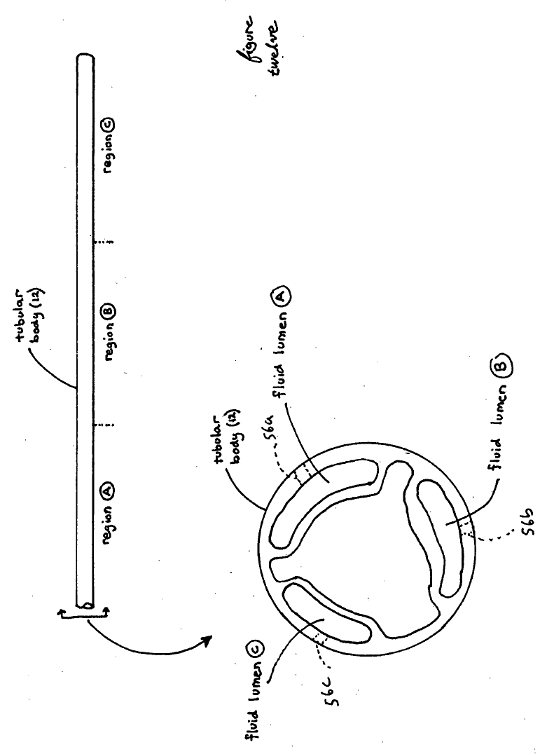

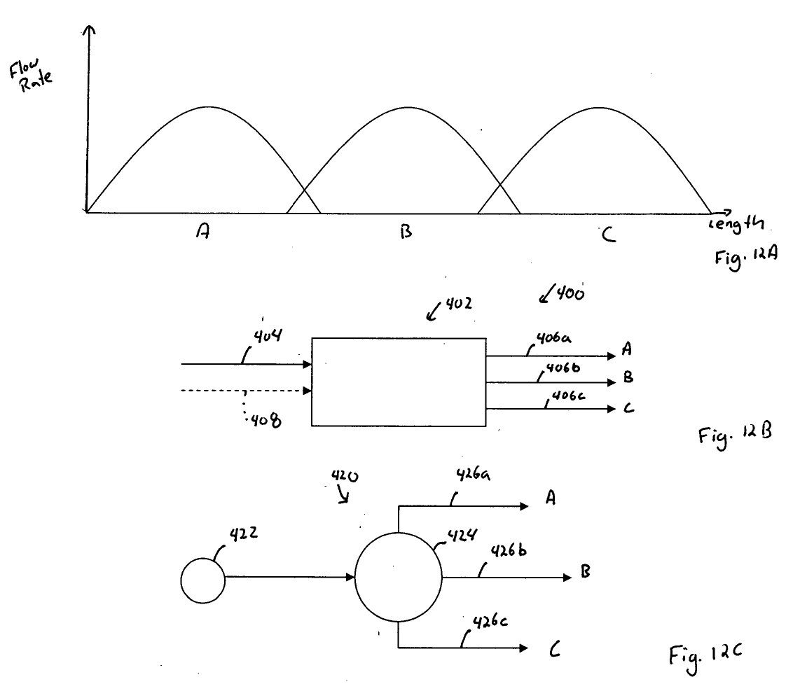

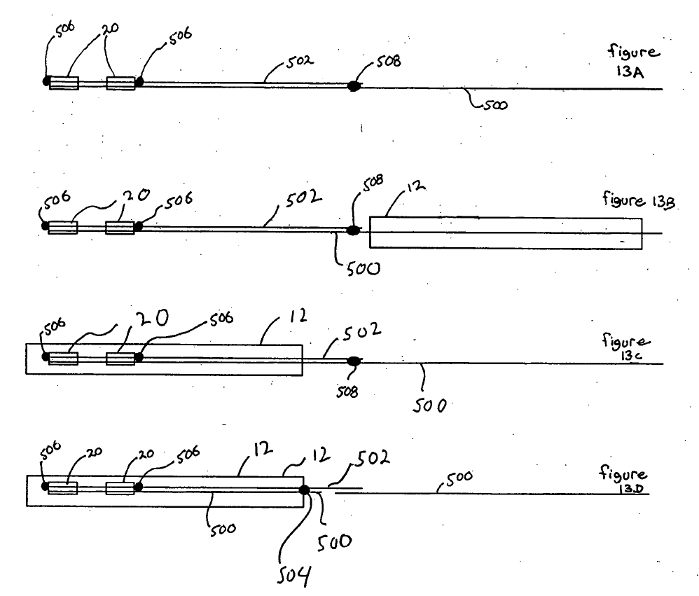

An exemplary embodiment for mounting one or more temperature sensors on or within a catheter is illustrated in FIGS. 13A through 13D. Such embodiments are particularly advantageous when used in combination with manufacturing ultrasonic catheters having multiple ultrasound radiating members, as disclosed herein. However, such embodiments are also particularly useful in combination with manufacturing other ultrasonic catheters, as well as with manufacturing other catheters that do not use ultrasound radiating members.

As illustrated in FIG. 13A, in an exemplary embodiment one or more temperature sensors 20 are coupled to a stiffening element 500, which is positioned in the catheter. As used herein, “temperature sensors” is defined broadly to include its ordinary meaning, as well as devices generally capable of measuring temperature, such as, for example, thermocouples. In an exemplary embodiment, the stiffening element 500 extends from the position of the temperature sensor 20 to the proximal end of the catheter. The stiffening element 500 is also optionally coupled to the catheter body 12. In this configuration, the stiffening element 500 limits axial movement between the temperature sensor 20 and the catheter body 12. Additionally, as will be explained herein, the stiffening element 500 is optionally also used to assemble and position the temperature sensor 20 within the catheter.

As in the exemplary embodiment illustrated in FIG. 8, the temperature sensor 20 and the stiffening element 500 (not illustrated in FIG. 8) are optionally positioned within one or more of the fluid delivery lumens 30. In a modified embodiment, the temperature sensor 20 and the stiffening element 500 are positioned one or more of the cooling fluid lumens 44. With respect to other catheter designs, the temperature sensor 20 and the stiffening element 500 are positionable within a variety of positions on or in the catheter, such as on a guidewire.

Referring again to FIG. 13A, in an exemplary embodiment, one or more temperature sensors 20 are coupled to the stiffening element 500. The stiffening element 500 is formable from a variety of materials, such as Nitinol or stainless steel. In one embodiment, the stiffening element 500 comprises a 304 stainless steel wire with a diameter of about 0.005 inches. In one embodiment, the temperature sensor 20 is coupled to the stiffening element 500 using an adhesive 506, such as cyanoacrylate disposed inside a polyimide sleeve. Other adhesives can be used in other embodiments. As illustrated in FIG. 13A, a temperature sensor wire 502 is operatively connected to the temperature sensors 20, and optionally extends along a portion of the stiffening wire 500.

In an exemplary embodiment, during manufacture of an ultrasonic catheter, the temperature sensor wire 502 is temporarily coupled near or at its proximal end to the stiffening element 500 at bond 508. A suitable material for temporarily coupling the temperature sensor wire 502 to the stiffening element 500 at bond 508 includes, but is not limited to, epoxy adhesive. As illustrated in FIG. 13B, in an exemplary embodiment the stiffening element 500 is longer than the catheter body 12. As illustrated in FIG. 13C, after the temperature sensors 20 are coupled to the stiffening element 500, the temperature sensor wire 502 is threaded through a selected catheter lumen, such as a fluid delivery lumen 30 or a cooling fluid lumen 44, illustrated in FIG. 8. The proximal end of the stiffening element 500 is then adjusted to position the temperature sensors 20 at a desired axial position within the catheter body 12. In an exemplary embodiment, the proximal end of the stiffening element 500 is removed and the temperature sensor wire 502 is separated from the stiffening element 500 and is operatively coupled to a control cable at a proximal region of the catheter.

As illustrated in FIG. 13D, a seal 504 is optionally provided where the temperature sensor wire 502 and the stiffening element 500 exit the catheter body 12. In one embodiment, the seal 504 couples the stiffening element 500 to the catheter body 12. Suitable materials for the seal 504 include, but are not limited to, epoxy adhesive.

The exemplary embodiments illustrated in FIGS. 13A through 13D provide several advantages. For example, the stiffening element 500 helps to maintain the temperature sensors 20 substantially in their axial position with respect to a centerline of the catheter body 10 despite catheter elongation or length change during bending. This is particularly advantageous for the embodiments that use the inner core described herein. In such embodiments, the axial relationship between the temperature sensors 20 and the ultrasound radiating members within the ultrasonic core is substantially preserved. This relationship is particularly important in embodiments that use the ultrasonic transducer groups described herein.

For example, in one embodiment, a temperature sensor is to be positioned distal to and proximal to an ultrasound radiating member group, as illustrated in FIGS. 5 and 6. For example, in an embodiment using five ultrasound radiating member groups, six temperature sensors are used: one distal temperature sensor, four intermediate temperature sensors posited between the ultrasound radiating member groups, and one proximal temperature sensor. In such embodiments, the feedback control system 68 is optionally used to adjust power to each ultrasound radiating member group as described herein. The stiffening element 500 helps to maintain the axial, spaced-apart relationship between the ultrasound radiating member groups and the temperature sensors. In such embodiments, the temperature information from the temperature sensors more accurately represents temperatures associated with the ultrasound radiating member groups, notwithstanding bending and flexing of the catheter body 12.

In certain embodiments, a catheter includes a stiffening element and temperature sensor combination associated with one or more of the lumens 30, 44. In a modified embodiment, more than one stiffening element and temperature sensor combination is associated with a particular lumen. In still other embodiments, the stiffening element and temperature sensor combination is optionally used in combination with an ultrasonic catheter without a cooling fluid lumen 44 and/or a fluid delivery lumen 30.

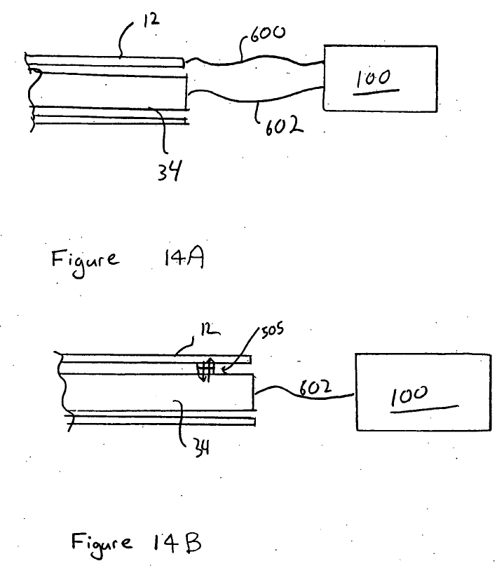

In certain embodiments, the catheter body 12 includes one or more temperature sensors that generate signals which are transmitted to the control system 100. In certain embodiments, the one or more temperature sensors receive signals which are generated by the control system 100. In one embodiment, the signals that are transmitted to and/or from the temperature sensors in the catheter body 12 are transmitted through a first control cable 600, as illustrated in FIG. 14A.

Similarly, in certain embodiments the inner core 34 includes one or more ultrasound radiating members that generate signals which are transmitted to the control system 100. In certain embodiments, the one or more ultrasound radiating members receive signals which are generated by the control system 100. In one embodiment, the signals that are transmitted to and/or from the ultrasound radiating members in the inner core 34 are transmitted through a second control cable 602, as also illustrated in FIG. 14A.

Referring now to the modified exemplary embodiment illustrated in FIG. 14B, the temperature sensors within the catheter body 12 are optionally operatively connected to the control system 100 through the inner core 34. In such embodiments, the signals from the temperature sensors in the catheter body are transmitted through the second control cable 602. This embodiment advantageously reduces the number of cables extending from the catheter 10 to the control system 100. Additionally, the catheter body 12 is not coupled to a control cable. This facilitates handling of the catheter body 12, which is typically delivered to the treatment site by itself over a guidewire. In such embodiments, the inner core 34, which is coupled to the first control cable 602, is inserted into the catheter body 12 once the catheter body 12 is properly positioned at the treatment site in the patient's vasculature. In a modified arrangement, the signals from the inner core are transmitted through the second control cable 604 that is associated with the catheter body 12. In yet another modified arrangement, more than one control cable extends from the inner core 34 to the control system.

A variety of connection devices 505 are usable to operatively connect the catheter body 12 to the inner core 34, and to allow signals to be transmitted and/or received through the second cable 602. Such configurations include, but are not limited to, spring or wire contacts, tabs, plugs and other configurations known in the electrical interfacing and wiring fields. In other embodiments, optical and/or electromagnetic connections are used.

In a modified embodiment, the inner core 34 and the catheter body 12 are operatively connected such that undesirable movement between the catheter body 12 and the inner core 34 is detectable. For example, in one embodiment, the inner core 34 is configured such that an electrical or other operative connection between the outer body 12 and the inner core 34 is achieved when the inner core 34 is properly positioned in the catheter body 12. When the inner core 34 is moved from the proper position, the connection is broken. The control system 100 can use the fact that the connection has been broken to generate an alarm or signal. In another embodiment, power to one more of the ultrasonic groups is be reduced or terminated when the connection is broken.

In still other embodiments, the inner core 34 and/or the catheter body 12 include various markers, such as metallic bands, which are sensed by a sensor on the other component. This configuration enables the control system to sense the position of the inner core 34 with respect to the catheter body 12 and to adjust the operating parameters of the catheter accordingly.

### SCOPE OF THE INVENTION

While the foregoing detailed description discloses several embodiments of the present invention, it should be understood that this disclosure is illustrative only and is not limiting of the present invention. It should be appreciated that the specific configurations and operations disclosed can differ from those described above, and that the methods described herein can be used in contexts other than treatment of vascular occlusions.

1. An ultrasound catheter configured to be positioned at a treatment site within a patient's vasculature, the catheter comprising:
an elongate tubular body forming a utility lumen;
an ultrasound assembly configured to be movably positioned within the utility lumen, wherein the ultrasound assembly includes a plurality of ultrasound radiating members; and
a plurality of fluid delivery lumens formed within the elongate tubular body, wherein each fluid delivery lumen includes one or more fluid delivery ports configured to allow a fluid to flow from within the fluid delivery lumen to the treatment site; wherein
a first fluid delivery lumen includes one or more fluid delivery ports over a first region of the tubular body, the first region having a distal end and a proximal end;
a second fluid delivery lumen includes one or more fluid delivery ports over a second region of the tubular body, the second region having distal end and a proximal end, and
a distance between the distal end of the first region and the proximal end of the second region being greater than a distance between the distal end of the first region and the proximal end of the first region.

2. The ultrasound catheter of claim 1, wherein the second region of the tubular body wholly includes the first region of the tubular body.

3. The ultrasound catheter of claim 1, wherein the first region of the tubular body and the second region of the tubular body do not overlap.

4. The ultrasound catheter of claim 1, further comprising:
a pump configured to provide fluid to one or more of the plurality of fluid delivery lumens; and
a valve assembly configured to selectively deliver fluid from the pump to one or more of the plurality of fluid delivery lumens.

5. The ultrasound catheter of claim 1, comprising a third fluid delivery lumen that includes one or more fluid delivery ports over a third region of the tubular body, the third region having distal end and a proximal end.

6. The ultrasound catheter of claim 5, wherein the first region is positioned distally from the second region, which is positioned distally of the third region.

7. The ultrasound catheter of claim 1, wherein the ultrasound radiating members are arranged in a plurality of electrical groups, such that a first group of the ultrasound radiating members can be separately activated with respect to a second group of the ultrasound radiating members.

8. The ultrasound catheter of claim 1, wherein:
the ultrasound radiating members are arranged in a plurality of electrical groups, such that a first group of the ultrasound radiating members can be separately activated with respect to a second group of the ultrasound radiating members;
the first group of the ultrasound radiating members are arrayed over a length of the ultrasound assembly that is substantially within the first region of the tubular body; and
the second group of the ultrasound radiating members are arrayed over a length of the ultrasound assembly that is substantially within the second region of the tubular body.

9. An ultrasound catheter comprising:
a tubular body;
an ultrasound assembly positioned within the tubular body; and
a plurality of fluid delivery lumens formed within the tubular body, wherein each fluid delivery lumen includes one or more fluid delivery ports configured to allow a fluid to flow from within the delivery lumen to the treatment site; wherein
a first fluid delivery lumen includes one or more fluid delivery ports along a first region of the tubular body,
a second fluid delivery lumen includes one or more fluid delivery ports along a second region of the tubular body, and
the first region includes a portion of the tubular body that is not included in the second region.

10. The ultrasound catheter of claim 9, wherein the ultrasound radiating members are arranged in a plurality of electrical groups, such that a first group of the ultrasound radiating members can be separately activated with respect to a second group of the ultrasound radiating members.

11. The ultrasound catheter of claim 9, wherein
the ultrasound radiating members are arranged in a plurality of electrical groups, such that a first group of the ultrasound radiating members can be separately activated with respect to a second group of the ultrasound radiating members; and
the first group of the ultrasound radiating members are arranged over a length of the ultrasound assembly is substantially corresponds in position to the first region of the tubular body.

12. The ultrasound catheter of claim 9, wherein the second region of the tubular body wholly includes the first region of the tubular body.

13. The ultrasound catheter of claim 9, wherein the first region of the tubular body and the second region of the tubular body do not overlap.

14. The ultrasound catheter of claim 9, further comprising:
a fluid reservoir hydraulically connected to a proximal region of the catheter; and
a valving assembly configured to selectively deliver fluid stored in the fluid reservoir to one or more of the plurality of fluid delivery lumens.

15. The ultrasound catheter of claim 9, further comprising:
a pump configured to deliver substantially constant flow of fluid; and
a valving assembly configured to alternately deliver fluid delivered by the pump to the first and second fluid delivery lumens.

16. The ultrasound catheter of claim 9, wherein three fluid delivery lumens are formed within the tubular body.

17. A method of treating a blockage within a patient's vasculature, the method comprising:
positioning an ultrasound catheter at the treatment site;
in a first treatment phase, delivering a therapeutic compound and ultrasonic energy from a first portion of the ultrasound catheter, such that at least a portion of the blockage is exposed to the therapeutic compound and the ultrasonic energy, and wherein the delivery of therapeutic compound and ultrasonic energy is configured to reduce the blockage;
monitoring progression of the blockage reduction;
in a second treatment phase, delivering a therapeutic compound and ultrasonic energy from a second portion of the ultrasound catheter, wherein the second portion of the catheter includes a catheter region that is not included in the first portion of the catheter.

18. The method of claim 17, wherein positioning the ultrasound catheter at the treatment site comprises passing at least a portion of the ultrasound catheter through the blockage.

19. The method of claim 17, wherein:
the therapeutic compound is delivered through a first fluid delivery lumen during the first treatment phase; and
the therapeutic compound is delivered through a second fluid delivery lumen during the second treatment phase.

20. A catheter system for delivering ultrasonic energy and a therapeutic compound to a treatment site within a body lumen, the catheter system comprising:
an elongate tubular body having an energy delivery section, the tubular body defining a utility lumen;
a fluid delivery lumen extending through at least a portion of the tubular body and having at least one fluid delivery port in the energy delivery section;
an ultrasound assembly configured to be inserted into the utility lumen, wherein the ultrasound assembly includes at least one ultrasound radiating member;
a stiffening element positioned in the tubular body; and
a temperature sensor coupled to the stiffening element.

21. The catheter system of claim 20, wherein the temperature sensor is a thermocouple.

22. The catheter system of claim 20, wherein the stiffening element is positioned within the fluid delivery lumen.

23. The catheter system of claim 20, wherein the stiffening element is positioned within the utility lumen.

24. The catheter system of claim 20, wherein a plurality of temperature sensors are coupled to the stiffening element.

25. The catheter system of claim 20, further comprising a temperature sensor wire that is electronically coupled to the temperature sensor and that is mechanically coupled to the stiffening element.

26. The catheter system of claim 25, wherein the stiffening element is longer than the elongate tubular body.

27. The catheter system of claim 26, wherein the stiffening element protrudes from a proximal end of the elongate tubular body.

28. A catheter system comprising:
an elongate tubular body having an energy delivery section, wherein the tubular body defines a utility lumen passing through the tubular body;
a fluid delivery lumen extending through at least a portion of the tubular body and having at least one fluid delivery port in the energy delivery section;
an ultrasound assembly configured for insertion into the utility lumen, wherein the ultrasound assembly includes at least one ultrasound radiating member;
a temperature sensor coupled to the elongate tubular body;
a control box containing control circuitry to control the ultrasound radiating members based on signals received form the temperature sensor; and
an electrical connection between the tubular body and the ultrasound assembly, the electrical connection configured to allow electronic signals to be passed between the tubular body and the ultrasound assembly.

29. The catheter system of claim 28, further comprising a cable connecting the ultrasound assembly with the control box, wherein the electrical connection between the control box and the tubular body is via the ultrasound assembly.

30. The catheter system of claim 28, further comprising a cable connecting the tubular body with the control box, wherein the electrical connection between the control box and the ultrasound assembly is via the tubular body.

31. The catheter system of claim 30, further comprising a position sensor coupled to the ultrasound assembly, wherein the position sensor is configured to detect a relative poison of the ultrasound assembly and the tubular body.
